(19) United States
(12) Patent Application Publication
(10) Pub. No.: US 2015/0322508 A1
(43) Pub. Date: Nov. 12, 2015

(54) METHOD FOR COMPLETE TRACKING OF A SET OF BIOLOGICAL SAMPLES CONTAINING DNA OR RNA THROUGH MOLECULAR BARCODE IDENTIFICATION DURING LABORATORIAL WORKFLOW AND KIT FOR COLLECTING BIOLOGICAL SAMPLES CONTAINING DNA OR RNA

(71) Applicant: FLEURY S.A., Jabaquara, São Paulo-SP, CEP: (BR)

(72) Inventors: Miguel MITNE NETO, São Paulo (BR); Kozue MIYASHIRO, São Paulo (BR); Marcos Tadeu DOS SANTOS, São Paulo (BR)

(73) Assignee: FLEURY S.A., Sao Paulo (BR)

(21) Appl. No.: 14/758,401

(22) PCT Filed: Dec. 28, 2012
(86) PCT No.: PCT/BR2012/000548

§ 371 Date: Jun. 29, 2015

(51) Int. Cl. C12Q 1/68

(52) U.S. Cl. CPC C12Q 1/6874

(57) ABSTRACT

The present invention discloses a method for complete tracking of a set of biological samples containing DNA or RNA through the use of molecular barcode identification during the course of laboratorial workflow, wherein the sample is contacted with primers comprising a molecular barcode and a universal primer sequence immediately after its collection and the amplification of the target regions occurs concomitantly to the barcode insertion, in one or both ends of said target region, in the same annealing temperature through a one step PCR. Finally, the invention also discloses kits for collecting biological samples containing DNA or RNA.

### FIELD OF THE INVENTION

The invention relates to a method for complete tracking of multiple samples during laboratorial workflow by adding a molecular barcode to target sequences through a one-step polymerase chain reaction (PCR). This one-step PCR method for tracking multiple biological samples permits an early and reliable identification of biological samples requiring only a very small amount of sample.

### BACKGROUND OF THE INVENTION

The development of the sequencing analysis technology in the field of molecular biology has shown to be a key tool in the researching of all branches of biological research. More specifically in the medical area, sequencing analysis plays an important role in the diagnosis and prognosis of genetic disorders. These diagnoses have been performed much more effectively over time with the development of newer and faster sequencing methodologies and equipments.

The automated Sanger sequencing has been the most important sequencing method in the scientific area for almost two decades (Metzker, M. Sequencing technologies—the next generation. Nature reviews Genetics (11). 2010). The completion of the only finish-grade human genome sequence, among other relevant accomplishments, was possible due to the Sanger sequencing.

Recently, new methodologies of sequencing have been developed. These technologies called next-generation sequencing (NGS) have the capacity of producing huge amounts of data in a fast and cheap manner, consisting basically of the steps of template preparation, sequencing and data analysis (Metzker, M. Sequencing technologies—the next generation. Nature reviews Genetics (11). 2010).

NGS can sequence up to billions of bases in a single day at a low cost (Pop, M. & Salzberg, S. Bioinformatics challenges of new sequencing technology. Trends Genet. 24(3). 2008). Although achieving an increasingly higher throughput is the ultimate goal of NGS technology, this increasingly higher throughput also presents considerable challenges for the technology.

In this sense, the massive amount of data produced by NGS methodologies place substantial demands on the development of more efficient routines in terms of data storage, tracking and quality control.

New findings in the medical area relate to common disorders, as cancer, phenylketonuria, among others, with mutations in the human genome. Therefore, the diagnosis and prognosis of such disorders benefits from sequencing methodologies advancements. Currently, laboratory processes involved in molecular diagnosis are highly complex and have proven to be difficult to standardize and automate (Gomah, M. et al. Modeling Complex Workflow in Molecular Diagnostics. The Journal of Molecular Diagnostics 12(1). 2010). The throughput of many samples brings an extra challenge; namely, the exchange of samples when analyzing a considerable number of experiments. Errors in tracking and in exchanging samples result in costly accidents in a clinical laboratory workflow, implications for patients, laboratories and operators.

Laboratorial tests, particularly in this biotechnological field, must therefore be performed under a high quality control system that is able to easily and accurately identify biological samples over the entire lifetime of the biological sample in the laboratory. However, particularly when considerably high numbers of samples are generated, the workflow becomes increasingly costly and complex. Therefore, large investments are necessary to maintain a structure in high quality standards.

The common identification system used in laboratories consists of labeling the sample contained in test tubes. Although this routine is very useful, the object being tracked is a tube or recipient and not specifically the analyte, the specific biological or chemical material contained by the tube, per se. Therefore, this laboratorial routine of tracking samples only by labeling the test tubes does not exclude the possibility of human mistakes during the manipulation of the samples over the laboratory workflow.

In this sense, molecular barcodes are an option that may be used in order to track analytes during molecular assay workflows, since they represent an entity with similar properties to the analyte. Molecular barcodes are, simply put, unique molecules used to identify a particular sample in a pool of samples. And in this present case may be inserted directly within the biologic material being analyzed so as to easily and rapidly identify its contents.

US 2010/0227329 A1 discloses processes of tagging a DNA target, through a two-step PCR reaction, using two different annealing temperatures for primers and barcodes and in the presence of linker molecules, such as avidin or streptavidin, to link functional molecules and core molecules.

WO 2010/115154 relates to a multi-step barcoding method directed towards target nucleic acids, wherein three primers are employed: reverse and forward amplification primers linked to a nucleotide tag and a barcode primer linked to a tag-specific portion.

WO 2012/019765 also discloses a method for combinatorial tracking of samples for parallel analysis and in particular a method for randomly mixing members of different libraries, each with a unique identifier. It is also disclosed that the insertion of the specific primers and barcodes occurs in different annealing temperatures; hence, making use of a two-step PCR reaction.

These documents show strategies for barcode insertions after the genetic material extraction; however, the documents cited do not adequately resolve the aforementioned problems regarding the elimination of errors as characterized by the traditional labeling of test tube collections, due in large part to the number of steps and subsequent transfers of the sample material before being reliably labeled.

In this sense, it is crucial to develop methods aiming at reducing the number of steps in labeling sample material over the course of the laboratory workflow, particularly when preparing and tagging samples for routine sequencing analyses. Ideally, these methods would allow for complete sample tracking and concomitantly reduce the chances for error.

Accordingly, it follows that the present invention discloses a novel comprehensive single-step complete sample tracking process, allowing for biological sample tracking from the moment of the sample collection through the final analysis report, in which the genetic material extraction step can be omitted and in which the barcode insertion occurs in a single step polymerase chain reaction.

In this invention, barcodes and the universal primers are in contact with the sample since its first collection, thus providing a complete tracking thereof from the initial introduction of biological sample collection. Furthermore, in the one-step PCR reaction detailed in the present invention, the annealing of specific primers for amplification and the annealing of adapters for barcodes insertion occur at the same temperature; hence, there is no physical or time separation between sample and barcode during the entire process.

Thus, this invention brings as a benefit over the prior art the ability to track biological samples containing genetic material from the moment of the biological materials first collection through its final analysis and this is achieved by a single step polymerase chain reaction. In other words, this invention presents a means by which biological samples are rapidly and reliably identified over the course of a laboratory workflow, minimizing chances for errors to occur, and hence improving the efficiency of the laboratory workflow and the overall research and development involving biological samples.

Finally, as an additional benefit, a very small sample amount is required according to the present invention when compared to the state of art. For example, generally, the prior art methods require around 3 mL of blood through venous puncture, while the method of the present invention requires amounts as little as 2 μL of blood. Therefore, it is possible to obtain the biological sample through a simple finger prick blood collection followed by contacting the blood drop with a filter paper.

The possibility of using a very small sample amount is extremely important to improve the efficiency of the laboratorial workflow, in particular with respect to clinical analysis laboratories, as a result of a reduction in the time involved in collecting blood, the costs of the analyses to be conducted, as well as also increasing patients' comfort during this collection process.

### SUMMARY OF THE INVENTION

The present invention relates to a method of complete biological sample tracking. In particular, the analyte itself is tracked from the moment of its collection through the insertion of molecular barcodes and the amplification of the target sequence. The method allows for complete and reliable biological sample tracking, eliminating the chances of misidentifying the collected biological samples.

In certain embodiments, the invention discloses a method of complete sample tracking since its collection, wherein the molecular barcodes insertion occurs simultaneously to the amplification of the target sequence in a single PCR step.

In a certain embodiment, a sample is placed in a tube comprising an oligonucleotide consisting of a molecular barcode binding to universal primers. The following step comprises the removal of an aliquot and placing it in the PCR tube comprising oligonucleotides of a specific primer binding to the universal reverse and forward primers. Then, the amplification of the target regions occurs concomitantly to the barcode insertion in the same annealing temperature through a one step PCR, without the need of steps of initial extraction or purification prior to the sequencing analysis. However, if preferred, initial extraction or the purification steps can be performed.

Such strategy can be applied, but are not restricted, to diagnostic methods involving Sanger and next-generation sequencing (NGS), as for example, but not restricted to, specific or captured amplicons, transcriptomes, exomes, miRNomes and genomes.

The following figures are for illustrative purposes only and do not limit the invention described above in any way.

### BRIEF DESCRIPTION OF THE DRAWINGS

FIG. 1. Example of a sample tagged with barcode 1 for Sanger sequencing. Differences regarding oligonucleotides used for NGS are described in the text.

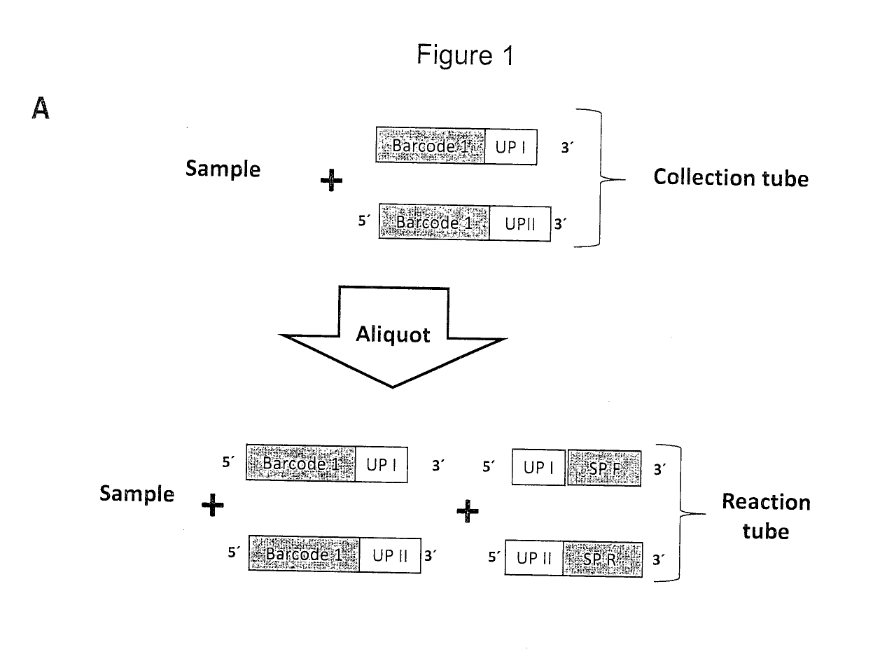

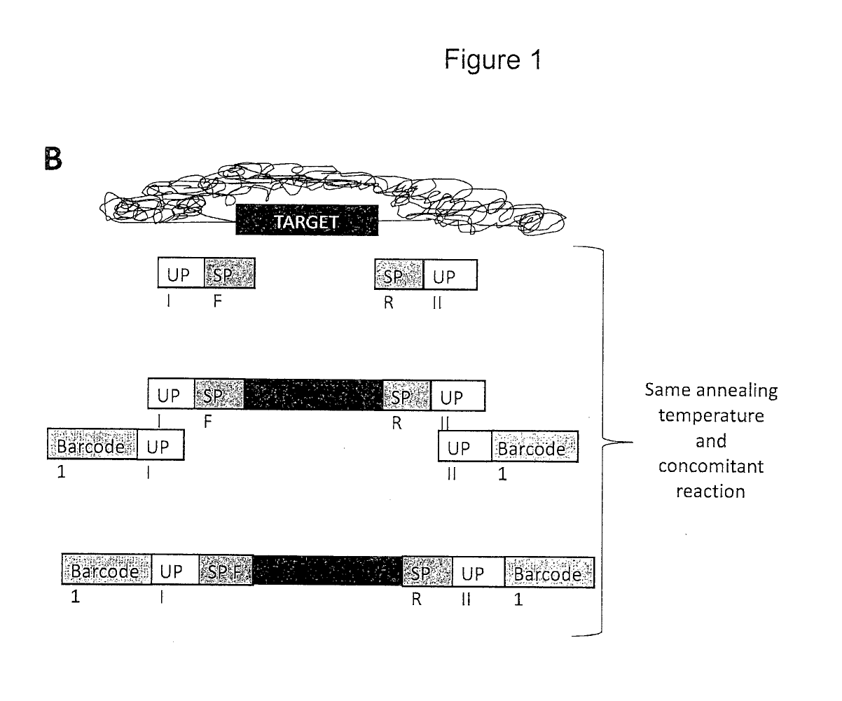

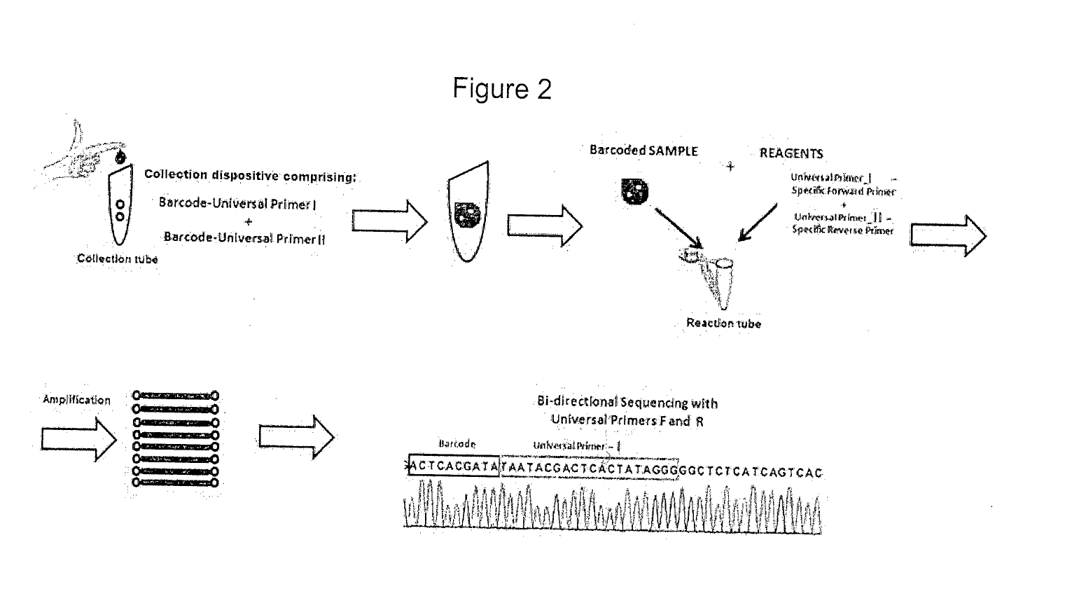

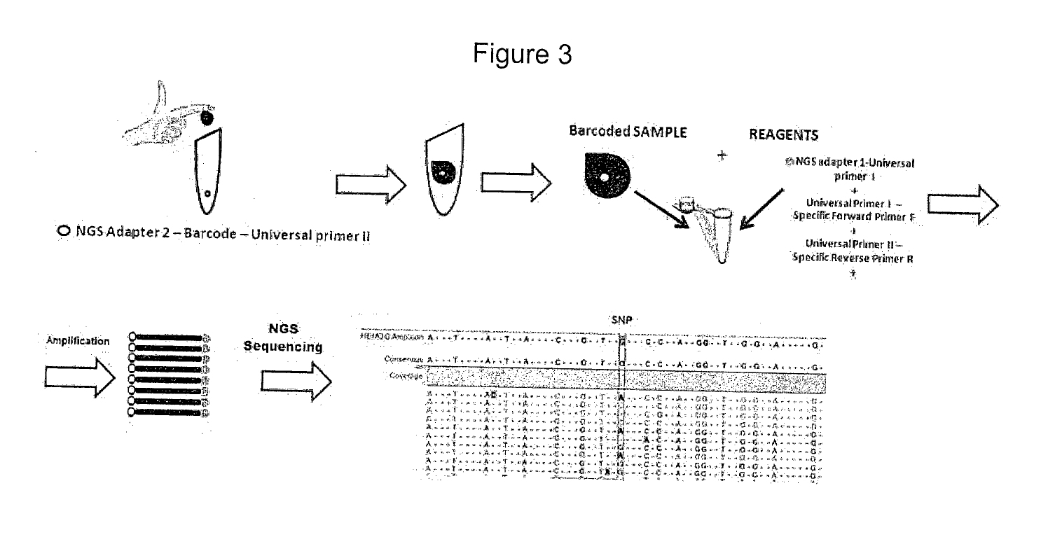

A) Sample barcoding workflow. The sample is collected in a tube containing oligonucleotides with the barcodes and universal primers I(UP I) and universal primers II (UP II). An aliquot of this mix (sample+barcode) is added to the PCR reagents (here shown only by the specific primers (SP F or SP R) with universal primers) making it possible to amplify the target region and insert the barcodes in a single PCR reaction.

B) Target region assembly. The target region is amplified by the annealing of the specific primers, which contain universal primers. The annealing of specific primers and universal primers in tandem with barcode sequences occurs at the same temperature and time, differently from the state of art, in which the annealing temperatures are different and the PCR occurs in two steps.

FIG. 2. Sanger sample barcoding overview: a pair of oligonucleotides (barcode-universal primer F and R) is already inside of the collection dispositive. Following the diagnostic workflow, an aliquot of the sample containing the barcodes is used for amplification. This step allows the barcode's insertion on both extremities of the target region. Bi-directional sequencing with universal primers or with specific primers can detect target region variations and concomitantly identify the respective barcode.

FIG. 3. Sample barcoding preparation for next generation sequencing. The method uses an oligonucleotide (here exemplified as NGS adapter 2-barcode-universal primer II), which will be inserted into the generated target region. As it is described for Sanger Sequencing barcoding, the method uses a single step PCR reaction. Besides the specific primers F and R, one has to use an oligonucleotide containing an NGS adapter 1-universal primer sequence, in order to have a target region that can be sequenced in a NGS system. Only corrected target fragments can be sequenced, “NGS 1” and “NGS 2” adaptors need to be in the correct position and frame.

### DETAILED DESCRIPTION

The present invention describes a method to prepare a plurality of nucleic acids target sequences of a biological sample to, first, facilitate the analysis of the biological sample through the use of molecular methods, mainly by sequencing methodologies, and, second, to identify rapidly and reliably each biological sample in a sample pool.

In a preferred embodiment the oligonucleotides comprising the specific primer and universal primer as well as the barcode and universal primer, respectively, are linked to the target sequence in a one step polymerase chain reaction (PCR), in which the annealing temperature is the same for both oligonucleotides.

Therefore, the embodiments disclosed by the present invention, as well the advantages over the state of art must be read concomitantly with the examples and figures also disclosed by the present invention.

The term “universal primers” refers to any sequences known to anneal to target sequences that do not interfere in the amplification of targeted genome/transcriptome/exome/miRNome region, either among themselves.

The terms “one step PCR” means a polymerase chain reaction in which the annealing temperature of the two sets of primers (barcode and universal primers as well as specific primers and universal primers) is the same and the reactions occurs at the same time.

The term “collection tube” means the first recipient where the biological sample is conditioned after its collection.

The term “barcode” means an oligonucleotide present in a nucleic acid sequence in order to identify it. Such identification is made through different laboratorial approaches.

In one embodiment of the invention, it is provided for a method that allows for the complete tracking of a set of biological samples containing DNA or RNA through molecular barcode identification during laboratorial workflow comprising the following steps:

i) contacting molecular barcodes and universal primers with the biological samples containing DNA or RNA in a collection means, thus forming mix A;

ii) contacting mix A with a PCR mix comprising specific primers and universal primers, thus forming mix B;

iii) amplifying the target region of the biological samples containing DNA or RNA and inserting the molecular barcodes in one or both ends of said target region in a single and concomitant step by performing a unique multiplex one step PCR in mix B;

iv) analyzing the product obtained in step iii) by any molecular technology.

In a preferred embodiment, the molecular barcodes are synthesized, or generated through molecular biology methodologies, in tandem with the universal primers.

In a further embodiment, the molecular barcodes are from 4 to 30 nucleotides long, preferably composed between 8 to 12 nucleotides and most preferably 10 nucleotides long.

In another embodiment, the volume of said biological samples containing DNA or RNA is from 0.5 μL to 5 mL, more preferably from 1 μL to 10 μL and most preferably 2 μL.

In a further embodiment, the annealing temperatures for amplification and for barcode insertion in the multiplex one step PCR of step iii) are held constant.

In a preferred embodiment, the annealing temperature is between 35° C. to 80° C., more preferably between 45° C. and 70° C., and most preferably 56° C.

In a further embodiment, the number of cycles of the multiplex one-step PCR of step iii) is between 10 and 60 cycles, more preferably between 20 and 50 cycles, and most preferably 45 cycles.

The method of the present invention is useful for a series of different biological samples from human or any other eukaryotic or prokaryotic organisms selected from the group consisting of blood and other fluids, paraffin or other forms of fixed tissues, cryopreserved samples, cultured cells, tissues, seeds, leaves, exudates, lavages and swabs material.

In a preferred embodiment, the biological sample containing DNA or RNA is blood.

In another embodiment, the blood is contacted with anticoagulants selected from the group consisting of EDTA, heparin or citrated-based anticoagulants.

The method of the present invention is also useful for a series of different synthetic samples that can be produced through in vivo or in vitro strategies.

In a further embodiment, the universal primers of steps i) and ii) are universal primers M13 and T7.

In a preferred embodiment, the molecular technology is a sequencing methodology.

In an even more preferred embodiment, the sequencing methodology is Sanger or Next-Generation Sequencing.

In a further embodiment, the collection means is selected form the group consisting of tubes, recipients, plates, papers, swabs and pipettes.

In a further embodiment, the molecular barcodes and universal primers are already present in the collection means in step (i).

In a further embodiment, step (i) is carried out immediately after the collection of said biological samples containing DNA or RNA.

In a further embodiment, the steps (i) and (ii) are carried out simultaneously.

In another embodiment, step (ii) is carried out after step (i) in a reaction tube or recipient.

The invention also discloses a method for complete tracking a set of biological samples containing DNA or RNA through molecular barcode identification during laboratorial workflow, comprising the following steps:

i) contacting molecular barcodes and universal primers with the biological samples containing DNA or RNA in a collection means, thus forming mix A;

ii) transferring an aliquot of mix A to a reaction tube or recipient wherein said reaction tube or recipient comprises PCR mix comprising specific primers and universal primers, thus forming mix B;

iii) amplifying the target region of the biological samples containing DNA or RNA and inserting the molecular barcodes in one or both ends of said target region in a single and concomitant step by performing a unique multiplex one-step PCR in mix B using the same annealing temperature for amplification and for barcode insertion;

iv) analyzing the product obtained in step (iii) by any molecular technology.

Furthermore, the invention also discloses a method for complete tracking of a set of biological samples containing DNA or RNA through molecular barcode identification during laboratorial workflow, comprising the following steps:

i) adding biological samples containing DNA or RNA to a collection means containing molecular barcodes and universal primers, thus forming mix A;

ii) transferring an aliquot of mix A to a reaction tube or recipient wherein said reaction tube or recipient comprises PCR mix comprising specific primers and universal primers, thus forming mix B;

iii) amplifying the target region of the biological samples containing DNA or RNA and inserting the molecular barcodes in one or both ends of said target region in a single and concomitant step by performing a unique multiplex one-step PCR in mix B using the same annealing temperature for amplification and for barcode insertion;

iv) analyzing the product obtained in step (iii) by any molecular technology.

One embodiment of the invention provides for a kit for collecting biological samples containing DNA or RNA comprising a set of collection tubes or recipients additionally comprising molecular barcodes, universal primers, specific primers and PCR mix in each collection tube or recipient for use in a method as disclosed by the present invention.

In a further embodiment, the invention also provides an additional kit for collecting biological samples containing DNA or RNA comprising a first set of collection tubes or recipients comprising molecular barcodes and universal primers in each collection tube or recipient, and a second set of reaction tubes or recipients comprising specific primers and PCR mix in each reaction tube or recipient, for use in a method as disclosed by the present invention.

### Biological Samples

The method of the present invention is useful for a series of different biological samples from human, other eukaryotic and prokaryotic organisms, or synthetic sequences generated through in vivo or in vitro strategies. Target samples could be, but are not restricted to any of the following: blood and other fluids, paraffin or other forms of fixed tissues, cryopreserved samples, cultured cells, tissues, seeds, leaves, exudates, lavages and swabs material.

The present invention provides the advantage of the collection of blood through a finger prick collection followed by contact of the blood drop with a filter paper or a small volume of anti-coagulant, since the blood volume used by the methodology can be very low.

In the case of blood samples, depending on the application it may be necessary that the collection tube comprises an anticoagulant. Examples of anticoagulants that can be used are those selected from the group consisting of EDTA, heparin or citrated-based anticoagulants.

### Sanger Sequencing Workflow

Before the material collection, sample tracking is done normally with the use of numeric barcodes and individual information in a proper system. In a preferred embodiment, the sample is collected using a collection dispositive containing the molecular barcodes. In a preferred embodiment a sample aliquot (with the oligonucleotide comprising molecular barcodes+universal primers) is added to lysis reagents. In a further embodiment, after lysis, an aliquot of this mixture (with the oligonucleotide comprising molecular barcodes+universal primers) is added to the PCR mix (among the components, the oligonucleotide comprising the specific primers+universal primers). The reaction occurs with the amplification of the target region followed by barcode insertion, without the need to stop the reaction and with the same annealing temperature for both oligonuleotides.

In a further embodiment, the target region is analyzed by Sanger sequencing procedures, which can be done by specific or universal primers. Barcodes are present at the end of each target region. Using bi-directional sequencing it is possible to guarantee the barcode presence in each of the reactions (forward and reverse).

In this sense, by the end of a laboratory workflow, the technician will be able to check the unique barcode against the sample number, thus proving that the reading result pertains to the referenced individual number upon collection and identifying the sample.

A simplified overview of the entire processes is shown in FIG. 2.

It is also important to note that the strategy described here can be applied if a genetic material extraction is required. The use of automated processes to perform an extraction does not restrict in anyway the invention. Finally, once the genetic material is collected, the sample is ready to be tagged, or identified, with a barcode.

### Next Generation Sequencing Workflow

In a further embodiment, the sample preparation for NGS purposes uses a very similar strategy as described for Sanger sequencing. However, for some NGS applications it is necessary to insert adapters at the end of each target region, in order to have a proper sample sequencing preparation. As an example, for use in the Ion PGM system, it is necessary the presence of adapters defined as “A” and “P1”. The oligonucleotides containing the adapter “A” and “P1” were synthesized in a format that the adapter is in the barcode 5′ end and the universal primers in the 3′ end, here described as M13 and T7. In this example, the oligonucleotide containing the sequence T7Universal Primer_Barcode_A-Adapter is, but is not restricted to, the sequence that must be present in the collection tube. Additionally, for this system, it is necessary to use an oligonucleotide synthesized as P1_universal primer, here exemplified as P1_M13, in order to have a feasible NGS sequencing target region fragment. The P1_M13 primer was added only at the PCR mix preparation, concomitantly with specific primers containing M13 and T7 sequences. For bi-directional sequencing, in this example, one should consider the use of adapters with M13 and T7 exchanged in described sequences.

At this moment, and for this example, one can expect the following fragments for the PGM system sequencing:

P1_M13_SpecificF_TARGET_REGION_SpecificR_T7_BARCODE_A_Adapter

P1_T7_SpecificF_TARGET_REGION_SpecificR_M13_BARCODE_A_Adapter

P1_M13_SpecificR_TARGET_REGION_SpecificF_T7_BARCODE_A_Adapter

P1_T7_{—} SpecificR_TARGET_REGION_SpecificF_M13_BARCODE_A_Adapter

After these steps target regions are treated as described in NGS workflow protocols. A simplified overview of the entire processes is shown in FIG. 3.

Table 1 shows examples of the oligonucleotides sequences used in the NGS methodologies described in this document. Specific primers sequences were designed to anneal to a portion of the human HFE gene.

TABLE 1

| | |
|---|---|
| M13HemocF | 5′TGTAAAACGACGGCCAGTCTGGATAACCTTGGC |
| specifc | TGTACC3′ |
| M13HemocR | 5′TGTAAAACGACGGCCAGTGGCTCTCATCAGTCA |
| specifc | CATACC3′ |
| T7HemocF | 5′TAATACGACTCACTATAGGGCTGGATAACCTTG |
| specifc | GCTGTACC3′ |
| T7HemocR | 5′TAATACGACTCACTATAGGG GGCTCTCATCAG |
| specifc | TCACATACC3′ |
| P1M13 | 5′CCTCTCTATGGGCAGTCGGTGATTGTAAAACGA |
| | CGGCCAGT3′ |
| P1T7 | 5′CCTCTCTATGGGCAGTCGGTGATTAATACGACT |
| | CACTATAGGG3′ |
| AdapA_BC1_M13 | 5′CCATCTCATCCCTGCGTGTCTCCGACTCAGACT |
| | CACGATATGTAAAACGACGGCCAGT3′ |
| AdapA_BC1_T7 | 5′CCATCTCATCCCTGCGTGTCTCCGACTCAGACT |
| | CACGATATAATACGACTCACTATAGGG3′ |
| AdapA_BC2_M13 | 5′CCATCTCATCCCTGCGTGTCTCCGACTCAGCGT |
| | GTCGCACTGTA AAACGACGGCCAGT3′ |
| AdapA_BC2_T7 | 5′CCATCTCATCCCTGCGTGTCTCCGACTCAGCGT |
| | GTCGCACTAAT ACGACTCACTATAGGG3′ |

### Reaction Conditions

A series of temperatures, enzymes and buffers were evaluated in order to achieve the amplification of a single genome region containing the molecular barcodes. The examples disclosed in the present invention are for illustrative purposes only and do not limit the invention in any way.

In a preferred embodiment the annealing temperature for amplification and barcode insertion is between 35° C. to 80° C. In a most preferred embodiment the annealing temperature is between 45° C. and 70° C. In an even more preferred embodiment the annealing temperature is 56° C.

In a preferred embodiment the number of cycles of the one step PCR is between 10 and 60 cycles. In a most preferred embodiment the number of cycles is between 30 and 50 cycles. In an even more preferred embodiment the number of cycles is 45.

In a preferred embodiment for a template it is used a preprepared mix from PROMEGA. Each reaction will contain two pairs of primers. In this example, one pair (M13 specific primer F+T7_specific primer R) will amplify the target region and the second pair, in this example, (Barcode_M13+Barcode_T7) will insert the barcode sequences. These two actions are performed with the same annealing temperature and in one step. FIG. 2 exemplifies this embodiment.

In a further preferred embodiment similar strategy of the above embodiment was used, however it was used a HotStarTaq (QIAGEN) reagents. Analogous conditions were used to prepare templates for NGS; however, this sequencing methodology use adapters and oligonucleotides as described above and in FIG. 3.

In the following examples two distinct reagents systems are used for amplification: (1) HotStarTaq enzyme (QIAGEN 203205 and the other components of the kit; (2) PROMEGA PCR MASTER MIX (catalog M7501). The parameters for each application are described in the respective sections.

The following examples are for illustrative purposes only and do not limit the invention described above in any way.

### EXAMPLE 1

A sample consisting of 2 μl of blood was transferred to a sample tube comprising anticoagulant (1 μl) and barcodes (0.2 μl in concentration of 100 μM). Then, 20 μl of lyse reagent of the Kit TaqMan Sample to SNP were added to the collection tube, followed by mixing the components in a vortex at room temperature for 5 minutes. Finally, 20 μl of DNA stabilization reagent of the Kit TaqMan Sample to SNP were also added to the sample tube.

Next, an aliquot of 10 μl of the resultant solution were removed and used in the amplification step of the target region in the HFE gene.

| | 1X | |
|---|---|---|
| MIX PROMEGA (2X) | 25 | μl |
| Specific Primer F (20 uM) | 0.3 | μl |
| Specific Primer R (20 uM) | 0.3 | μl |
| Sample aliquot | 10 | μl |

| | |
|---|---|
| DNAse/RNAse-Free water | Volume sufficient to 50 μl |

The specific primers used in the amplification of a region of HFE gene, in the chromosome 6, to detect the alteration c.845G>A (C282Y). were: F 5′CTGGATAACCTTGGCTGTACC3′ and R 5′GGCTCTCATCAGTCAC ATACC3′. These specific primers were designed so that in the end 5′ the sequence of the universal M13 (5′GTAAAACGACGGCCAGT3′) or T7 (5′TAATACGACTCACTATAGGG3′) were inserted.

In this step, the difference between the preparation for Sanger or NGS methods relies in the use of oligonucleotides containing adapters (“P1” and “A”) for the exemplified NGS method.

The amplification was performed in the thermocycler Veriti (Applied Biosystems—Life Technologies) and the cycling was the following:

95° C. —15 min

95° C. —30seg/56° C. —30seg/72° C. —30seg (45 cycles)

72° C. —10 min

4° C. —undetermined

After the amplification, the PCR products were purified using the GFX Kit (GE Healthcare 28903471) and quantified by spectrophotometry using Nanodrop 2000 (ThermoScientific). It is important to note that, depending on the particular optimization approaches, this step is optional.

For applications of Sanger sequencing it was used a standard protocol described in the kit BigDye Terminator v3.1 (Life Technologies), available on the company website and for NGS applications was used standard protocol for sequencing of target regions according to Ion PGM (Life Technologies).

### Example 2

A sample consisting of 2 μl of blood was transferred to a sample tube comprising anticoagulant (1 μl) and barcodes (0.2 μl in concentration of 100 μM). Then, 20 μl of lyse reagent of the Kit TaqMan Sample to SNP were added to the collection tube, followed by mixing the components in a vortex at room temperature for 5 minutes. Finally, 20 μl of DNA stabilization reagent of the Kit TaqMan Sample to SNP were also added to the sample tube.

Next, an aliquot of 10 μl of the resultant solution were removed and used in the amplification step of the target region in the JAK2 gene.

| | 1X | |
|---|---|---|
| Buffer (5X) | 5 | μl |
| dNTP Roche 12269023 (10 mM) | 1 | μl |
| QSolution (5X) | 5 | μl |
| Specific Primer F (20 μM) | 0.3 | μl |
| Specific Primer R(20 μM) | 0.3 | μl |
| Taq 5 U/μl | 0.5 | μl |
| Sample aliquot | 10 | μl |

| | |
|---|---|
| DNAse/RNAse-Free water | Volume sufficient to 50 μl |

The specific primers used in the amplification of a region of JAK2 gene, in the chromosome 9, to detect the alteration c. c.1849G>T (V617F) were: F 5′GCAGCAAGTATGATGAGCAAGCTT3′ and R 5′GGCATTAGAAAGCCTGTAGTTTTACTTAC3′. These specific primers were designed so that in the end 5′ the sequence of the universal M13 (5′TGTAAAACGACGGCCAGT3′) or T7 (5′TAATACGACTCACTATAGGG3′) were inserted.

In this step, the difference between the preparation for Sanger or NGS methods relies in the use of oligonucleotides containing adapters (“P1” and “A”) for the exemplified NGS method. The amplification was performed in the thermocycler Veriti (Applied Biosystems—Life Technologies) and the cycling was the following:

95° C. —15 min

95° C. —30seg/56° C. —30seg/72° C. —30seg (45 cycles)

72° C. —10 min

4° C. —undetermined

After the amplification, the PCR products were purified using the GFX Kit (GE Healthcare 28903471) and quantified by spectrophotometry using Nanodrop 2000 (ThermoScientific). It is important to note that, depending on the particular optimization approaches, this step is optional.

For applications of Sanger sequencing, it was used standard protocol described in the kit BigDye Terminator v3.1 (Life Technologies), available on the company website; and for NGS applications, it was used standard protocol for sequencing of target regions according to Ion PGM (Life Technologies).

1. A method for complete tracking of a set of biological samples containing DNA or RNA through the use of molecular barcode identification during the course of laboratorial workflow, characterized by comprising the following steps:
i) contacting molecular barcodes and universal primers with the biological samples containing DNA or RNA in a collection means, thus forming mix A;
ii) contacting mix A with a PCR mix comprising specific primers and universal primers, thus forming mix B;
iii) amplifying the target region of the biological samples containing DNA or RNA and inserting the molecular barcodes in one or both ends of said target region in a single and concomitant step by performing a unique multiplex one-step PCR in mix B;
iv) analyzing the product obtained in step iii) by any molecular technology.

2. The method according to claim 1, characterized in that, in step i), the molecular barcodes are synthesized, or generated through molecular biology methodologies, in tandem with the universal primers.

3. The method according to claim 2, characterized in that the molecular barcodes are from 4 to 30 nucleotides long, preferably composed between 8 to 12 nucleotides and most preferably 10 nucleotides long.

4. The method according to any one of claims 1 to 3, characterized in that the volume of said biological samples containing DNA or RNA is from 0.5 μL to 5 mL, more preferably from 1 μL to 10 μL and most preferably 2 μL.

5. The method according to any one of claims 1 to 4, characterized in that the annealing temperatures for amplification and for barcode insertion in the multiplex one-step PCR of step iii) are held constant.

6. The method according to claim 5, characterized in that the annealing temperature is between 35° C. to 80° C., more preferably between 45° C. and 70° C., and most preferably 56° C.

7. The method according to any one of claims 1 to 6, characterized in that the number of cycles of the multiplex one-step PCR of step iii) is between 10 and 60 cycles, more preferably between 20 and 50 cycles, and most preferably 45 cycles.

8. The method according to any one of claims 1 to 7, characterized in that it is useful for a series of different biological samples from human or any other eukaryotic and prokaryotic organisms selected from the group consisting of blood and other fluids, paraffin or other forms of fixed tissues, cryopreserved samples, cultured cells, tissues, seeds, leaves, exudates, lavages and swabs material.

9. The method according to claim 8, characterized in that the biological sample containing DNA or RNA is blood.

10. The method according to claim 9, characterized in that, the blood is contacted with anticoagulants selected from the group consisting of EDTA, heparin or citrated-based anticoagulants.

11. The method according to any one of claims 1 to 10, characterized in that it is useful for a series of different synthetic samples that can be produced through in vivo or in vitro strategies.

12. The method according to any one of claims 1 to 11, characterized in that the universal primers of steps i) and ii) are universal primers M13 and T7.

13. The method according to any one of claims 1 to 12, characterized in that the molecular technology is a sequencing methodology.

14. The method according to claim 13, characterized in that the sequencing methodology is Sanger or Next-Generation Sequencing.

15. The method according to any one of claims 1 to 14, characterized in that the collection means is selected from the group consisting of tubes, recipients, plates, papers, swabs and pipettes.

16. The method according to any one of claims 1 to 15, characterized in that the molecular barcodes and universal primers are already present in the collection means in step i).

17. The method according to anyone of claims 1 to 16, characterized in that step i) is carried out immediately after the collection of said biological samples containing DNA or RNA.

18. The method according to anyone of claims 1 to 17, characterized in that steps i) and ii) are carried out simultaneously.

19. The method according to anyone of claims 1 to 17, characterized in that step ii) is carried out after step i) in a reaction tube or recipient.

20. A method for complete tracking of a set of biological samples containing DNA or RNA through the use of molecular barcode identification over the course of laboratorial workflow, characterized by comprising the following steps:
i) contacting molecular barcodes and universal primers with the biological samples containing DNA or RNA in a collection means, thus forming mix A;
ii) transferring an aliquot of mix A to a reaction tube or recipient wherein said reaction tube or recipient comprises PCR mix comprising specific primers and universal primers, thus forming mix B;
iii) amplifying the target region of the biological samples containing DNA or RNA and inserting the molecular barcodes in one or both ends of said target region in a single and concomitant step by performing a unique multiplex one-step PCR in mix B using the same annealing temperature for amplification and for barcode insertion;
iv) analyzing the product obtained in step iii) by any molecular technology.

21. A method for complete tracking of a set of biological samples containing DNA or RNA through the use of molecular barcode identification over the course of laboratorial workflow, characterized by comprising the following steps:
i) adding biological samples containing DNA or RNA to a collection means containing molecular barcodes and universal primers, thus forming mix A;
ii) transferring an aliquot of mix A to a reaction tube or recipient wherein said reaction tube or recipient comprises PCR mix comprising specific primers and universal primers, thus forming mix B;
iii) amplifying the target region of the biological samples containing DNA or RNA and inserting the molecular barcodes in one or both ends of said target region in a single and concomitant step by performing a unique multiplex one-step PCR in mix B using the same annealing temperature for amplification and for barcode insertion;
iv) analyzing the product obtained in step iii) by any molecular technology.

22. Kit for collecting biological samples containing DNA or RNA, characterized by comprising a set of collection tubes or recipients comprising molecular barcodes, universal primers, specific primers and PCR mix in each collection tube or recipient for use in a method as defined in any one of claims 1 to 19.

23. Kit for collecting biological samples containing DNA or RNA, characterized by comprising a first set of collection tubes or recipients comprising molecular barcodes and universal primers in each collection tube or recipient, and a second set of reaction tubes or recipients comprising specific primers and PCR mix in each reaction tube or recipient, for use in a method as defined in claim 20 or 21.
